(19) United States
(12) Patent Application Publication
(10) Pub. No.: US 2015/0342560 A1
(43) Pub. Date: Dec. 3, 2015

(54) Novel Algorithms for Feature Detection and Hiding from Ultrasound Images

(71) Applicant: ULTRASAFE ULTRASOUND LLC, Gaithersburg, MD (US)

(72) Inventors: Sonya DAVEY, Gaithersburg, MD (US); Haris Godil, Boyds, MD (US); Abhishek Biswas, Calcutta (IN); Samir Devalaraja, Acton, MA (US); Neil Davey, Gaithersburg, MD (US); Raj Shekhar, Gayton, MD (US)

(21) Appl. No.: 14/652,459

(22) PCT Filed: Jan. 27, 2014
(86) PCT No.: PCT/US2014/013215

§ 371 Date: Jun. 16, 2015

Related U.S. Application Data
(60) Provisional application No. 61/756,482, filed on Jan. 25, 2013.
(60) Provisional application No. 61/768,553, filed on Feb. 25, 2013.
(60) Provisional application No. 61/862,512, filed on Aug. 5, 2013.

(51) Int. Cl. A61B 8/08; A61B 8/00

(52) U.S. Cl. CPC A61B 8/085; A61B 8/0866; A61B 8/461; A61B 8/5215; A61B 8/5207

(57) ABSTRACT

An embodiment that relates to a method for detecting a fetus genitalia in an ultrasound image, having excluding regions from the ultrasound image, wherein the regions do not contain any feature of the fetus genitalia; identifying points of interest (POIs) from remaining regions of the ultrasound image; detecting the fetus genitalia from the POIs.

### RELATED APPLICATIONS

This application is a U.S. National Phase filing under 35 USC 371 of PCT/US14/13215, filed Jan. 27, 2014 claiming priority under 35 USC 119(e) from U.S. Provisional Patent Application Ser. Nos. 61/756,482, filed Jan. 25, 2013; 61/768,553, filed Feb. 25, 2013; and 61/862,512, filed Aug. 5, 2013. These provisional applications and all other U.S. patents and applications, both provisional and non-provisional, cited in this application are incorporated herein in their entirety by reference.

### FIELD OF INVENTION

This invention is related to novel algorithms for feature detection and hiding information from ultrasound images. The novel algorithms could be incorporated into ultrasound software or hardware. The algorithm can efficiently and accurately locate and hide the genitalia in live images produced by ultrasound machines. This technology, when incorporated into ultrasound machines, could reduce the problem of female feticide that is rising throughout the world.

### BACKGROUND

Over 163 million females have been selectively aborted since 1980 and around 5.3 million female-selective abortions occur each year. Sex-selective abortion occurs at highest rates in China, Armenia, India, Albania, Vietnam, Azerbaijan, Georgia, and South Korea. Currently, 45% of the world's population lives in societies with highly skewed male to female ratios, and 70% of the people in Asia live in such societies. Such an imbalance in sex ratios further decreases the status of women in society. In regions with extremely low levels of women (some as low as 500 females to 1000 males), young girls from other states are purchased as “wives.” Women are treated as a commodity, thus increasing their exploitation, sex trade, and rape.

95% of pre-natal sex-detection occurs via the use of ultrasound imaging. The increase in ultrasound portability, decrease in cost, and increase in accuracy has led to an implosion of private clinics able to determine sex in India. A study in Haryana, India shows although there is a “felt need” to have a male child, this “need” does not translate into female feticide unless it is aided by technology. Locations with a higher number of ultrasound machines also have an increased number of female feticides. In urban areas of India with a high density of ultrasound machines, there exists greater sex-imbalance in the population.

United Nations, many individual governments, and public health activists are actively searching for solutions to curb this genocide. Current solutions include governmental mandates prohibiting sex-selective abortions and organizational attempts to increase the status of the female child through education programs.

36 countries have already created strong governmental regulation to stop sex-selective abortion and curb female feticide. In addition to Asian countries with high sex ratio imbalances, 18 Western countries like the United Kingdom and Canada have also passed laws against sex-selective abortion. Due to the severity of the problem in India and China, both countries have passed laws that do not allow doctors to disclose the sex of the fetus. Additionally, all sex-determination technologies, except for ultrasound, are banned.

### BRIEF DESCRIPTION OF THE DRAWINGS

FIG. 1 shows a schematic diagram of the ultrasound-imaging algorithm in which the digital signals enter into the processor where the raw image is developed and this image is then checked whether it contains data regarding a feature such as the genitals of the fetus.

FIG. 2 shows a schematic diagram of the three stages for the identification of the genitals in ultrasound images.

FIG. 3 shows the region determined to be the location of the genitalia being automatically blacked out from the ultrasound image.

FIG. 4 shows a schematic diagram for nasal bone detection by a first embodiment.

FIG. 5 shows a surface topography map after convert the image correlation into surface plotting graph for the nasal detection according to the first embodiment.

FIG. 6 shows a schematic diagram for nasal bone detection by a second embodiment.

FIG. 7 shows a schematic diagram of stage 2 for the identification of the genitals.

FIG. 8 shows a schematic diagram of stage 3 for the identification of the genitals.

FIG. 9 shows the positive and possible negative training examples for the classifier.

FIG. 10 shows a schematic diagram of the ultrasound-imaging algorithm using the sliding window based approach.

FIG. 11 shows a flow diagram of the training of the classifier to recognize the difference between areas with and without genitalia.

FIG. 12 shows the sliding window method traversing through the image to locate areas that contain genitalia.

### SUMMARY OF THE INVENTION

Disclosed herein is a method for detecting a fetus genitalia in an ultrasound image, comprising: excluding regions from the ultrasound image, wherein the regions do not contain any feature of the fetus genitalia; identifying points of interest (POIs) from remaining regions of the ultrasound image; detecting the fetus genitalia from the POIs.

According to an embodiment, the regions excluded contain features of the fetus selected from a group consisting of skull, femur bone and nasal bone.

According to an embodiment, identifying POIs comprises selecting regions with a contrast above a threshold.

According to an embodiment, the method further comprises excluding POIs with curvatures below a threshold.

According to an embodiment, the method further comprises obscuring regions around the POIs from which the fetus genitalia is detected.

According to an embodiment, detecting the fetus genitalia from the POIs comprises detecting features characteristic of labia, scrotum, penis, or a combination thereof.

According to an embodiment, identifying POIs comprises selecting regions adjacent to pixels with the lowest 10% intensity among all pixels of the ultrasound image.

According to an embodiment, detecting the fetus genitalia from the POIs comprises using a classification model.

According to an embodiment, the method further comprises training a classification model using ultrasound images that contain at least one feature of fetus genitalia.

According to an embodiment, the method further comprises training a classification model using ultrasound images that do not contain any feature of fetus genitalia.

According to an embodiment, the classification model is a support vector machine.

According to an embodiment, the classification model is a sliding window classifier.

Disclosed herein is a sonographic instrument comprising a physical processor and a physical memory having instructions recorded thereon, the instructions when executed by the physical processor implementing any of the methods above.

According to an embodiment, the sonographic instrument further comprises a transducer configured to product an ultrasound wave, a sensor configured to receive echo of the ultrasound wave, and a display configured to display the ultrasound image.

Disclosed herein is a method for diagnosing anomaly in an ultrasound image, comprising: obtaining the ultrasound image from a patient; making a redacted image by obscuring at least one region of the ultrasound image; making information of the redacted image available to the patient; anonymizing the ultrasound image; diagnosing the anomaly by analysing the anonymized ultrasound image; making the diagnosis available to the patient; wherein no information of the obscured region except the diagnosis is made available to the patient.

Disclosed herein is a method comprising detecting a feature in an ultrasound image prior to displaying the feature on a monitor in a vicinity of an ultrasound machine, and hiding the feature so as to prevent the feature from being displayed on the monitor, the method employing a sliding window algorithm.

According to an embodiment, the method further comprises retaining the feature that is hidden from viewing on the monitor for remote viewing.

According to an embodiment, the method further comprises exploiting similarity between frames of ultrasound images close in time to transfer findings from one frame to another frame.

According to an embodiment, the method further comprises a graphics processing unit acceleration for real-time implementation.

### DETAILED DESCRIPTION

The novel technological solution of the present invention strives to automatically prevent ultrasound sex determination, thus decreasing sex-selective abortion. The technology provides ultrasound manufacturers a significant competitive advantage in ultrasound markets and a long-term insurance policy against criminal prosecution. In the highly competitive market of ultrasound technology in developing nations such as India, ultrasound manufacturers currently face significant societal backlash and numerous prosecutions.

The novel technological solution of the present invention also provides health professionals an insurance policy to avoid fetal sex determination and consequent criminal prosecution as laws in some countries such as India mandate that sex determination of the fetus is a crime.

In one embodiment, the algorithm employs a three-step process to locate the genitalia. In the first step, the algorithm searches for distinguishable characteristics of the fetus such as skull, femur bone, and nasal bone. The location and position of both the skull and one or both femur bones can significantly reduce the region of interest.

In the second step, the algorithm searches the region of interest and selects points of interest. The genitalia are visible in relief against the amniotic fluid, which is the lowest intensity uniform region in the ultrasound image. Thus, a distinguishing characteristic of pixels within the genitalia is their high intensity compared to pixels within the amniotic fluid. Pixels bordering the amniotic fluid (lowest intensity) will be selected as points of interest (POIs). A second distinguishing characteristic of pixels within the genitalia is high curvature, due to the structure of the genitalia. The algorithm will sample the contours in the image and calculate the curvature of each POI on the contours. Those POIs whose curvature fails to match that of genitalia are removed, leaving a new and reduced set of POIs.

In the final step, the algorithm searches for genitalia-related features at each POI. Genitalia will appear as either the “three-line sign” (females) or as a pair of elliptical regions of similar size (males). At each POI, the algorithm defines a region around the POI in which to search for genitalia-related features. The region determined to be the location of the genitalia will be automatically blacked out from the ultrasound image.

As the novel software of this invention interferes with only the images produced by an ultrasound system, it serves as “add on” software compatible with a wide variety of ultrasound machines. In other embodiments, security systems to ensure long-term implementation of the technology in ultrasound machines could be implemented. The security system could report tampering with the genital-blocking technology.

The novel algorithm for feature detection and hiding in ultrasound images could be implemented in ultrasound machine during original equipment manufacturing (OEM). Also, the novel algorithm could be retrofitted in existing ultrasound machines by charging health professionals, for example. To carry out the retrofitting, one could use existing distribution chains and maintenance personnel from manufacturers.

Alternatively, the novel algorithm could be used in a service business. For example, India's Pre-Natal Diagnostic Techniques (Regulation and Prevention of Misuse) Act, 1994, requires data retention. All records of pregnant women who have undergone an ultrasonography must be preserved for a period of two years. The PNDT (RPM) Rules, 1996 require that when the records are maintained on a computer, the person responsible for such record should preserve a printed copy of the record after authentication.

Furthermore, in one embodiment of the invention, one could address the problem of an abnormal growth in the region which is blurred out. The solution to this problem is that records of pregnant women who have undergone an ultrasonography (except the patient's name and address), including information regarding the regions blurred out and the surrounding region will be transmitted to a safe ultrasound image reviewing center with a code number (not patient's name or address) for each patient, and radiology technicians at the safe ultrasound image reviewing center will check for any abnormal growth. Then, the safe ultrasound image reviewing center will send a report to the doctor that conducted the ultrasound informing: (1) no abnormal growth or (2) possible abnormal growth, and patient needs further ultrasound testing which can then be done at secured and approved ultrasound facilities.

The above solution solves the problem of not detecting possible abnormal growth in the blurred region but also meets the data retention requirement under the Indian law, for example. The above requirement of sending information regarding each patient will automatically allow the safe ultrasound image reviewing center to keep track of the number of ultrasound exams performed in India, for example. Also, this solution creates a service business for the safe ultrasound image reviewing center with an ongoing revenue stream.

The post-monitoring of blurred out area and its surrounding of genital has several advantages: (1) the number of ultrasound exams done on pregnant mothers can be determined. This information is important for the government and world health organizations (note that the information will not be on the patients, thereby maintaining patient privacy); (2) the safe ultrasound image reviewing center will have a long-term and continued relationship with the doctors, allowing for future software updates to be provided to the doctors; and (3) the patients will get a second opinion on the ultrasound test with respect to the scan of the blurred out area and its surrounding.

### EXAMPLES

By producing software that has the ability to automatically detect and then blurred out the genital area of a fetus, one embodiment relates to a solution for female feticide in developing countries. FIG. 1 shows a schematic diagram of the ultrasound imaging algorithm in which the digital signals enter into the processor where the raw image is developed and this image is then checked whether it contains data regarding a feature such as the genitals of the fetus.

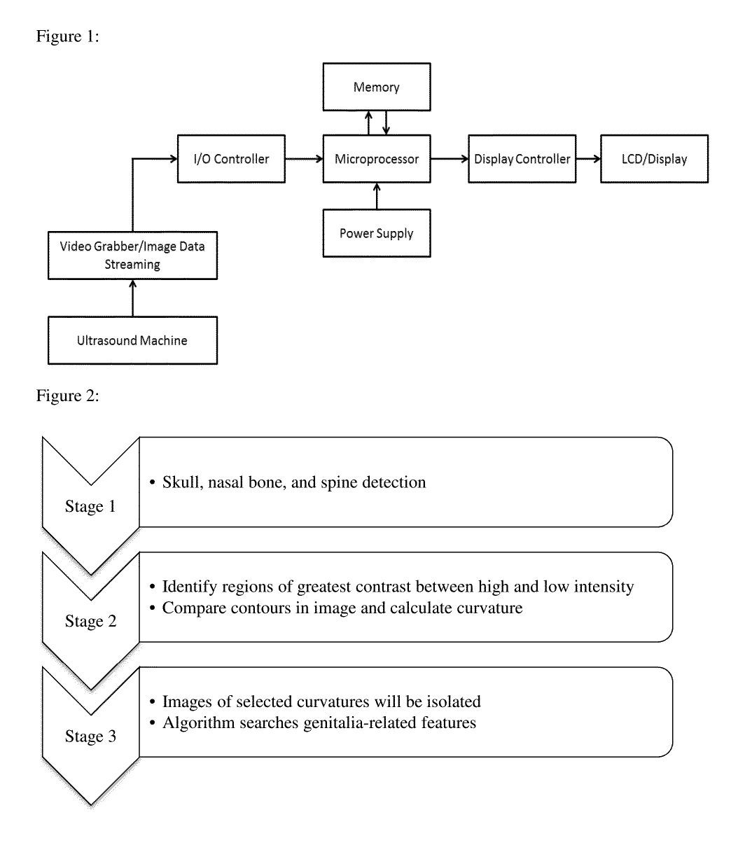

The identification of the genitals is done in three stages. FIG. 2 shows a schematic diagram of the three stages for the identification of the genitals in ultrasound images.

The algorithm employs a three-step process to locate the genitalia. In the first step, the algorithm searches for distinguishable characteristics of the fetus such as skull, femur bone, and nasal bone. Any detectable features are used to segment the fetus and narrow down the region of interest. In the second step, the algorithm performs a rough search over the region of interest and selects points of interest (POIs) based on the criteria described below. In the third step, the algorithm searches for the genitalia in the vicinity of each POI.

The skull and femur bones, if visible, are distinguishing characteristics of the fetus that can be used to reduce the region of interest. The skull appears as a relatively high-intensity ellipse or fragmented ellipse and can be detected using the iterative randomized Hough transform. The femur bone is also relatively high intensity and has a linear shape, making it detectable by line tracking. If the location and position of both the skull and one or both femur bones is known, the region of interest can be significantly reduced. Other features, such as the nasal bone, may also be detectable to further segment the fetus and narrow the region of interest.

In the second step, the algorithm searches the region of interest and selects points of interest. It is assumed that the genitalia are visible in relief against the amniotic fluid, which is the lowest intensity uniform region in the ultrasound image. Thus, a distinguishing characteristic of pixels within the genitalia is their high intensity compared to pixels within the amniotic fluid. Because different ultrasound images will not have the same intensities, the histogram of the image is computed using a stepwise linear function with the lowest 10% intensity pixels receiving an intensity of 0 and the highest 10% receiving an intensity of 255. Each pixel in the image at a specified sampling space will be analyzed based on the pixels surrounding it. Pixels bordering the amniotic fluid (lowest intensity) will be selected as POIs. A second distinguishing characteristic of pixels within the genitalia is high curvature, due to the structure of the genitalia. The algorithm will sample the contours in the image and calculate the curvature of each POI on the contours. Those POIs whose curvature fails to exceed a certain threshold will be thrown out, leaving a new and reduced set of POIs.

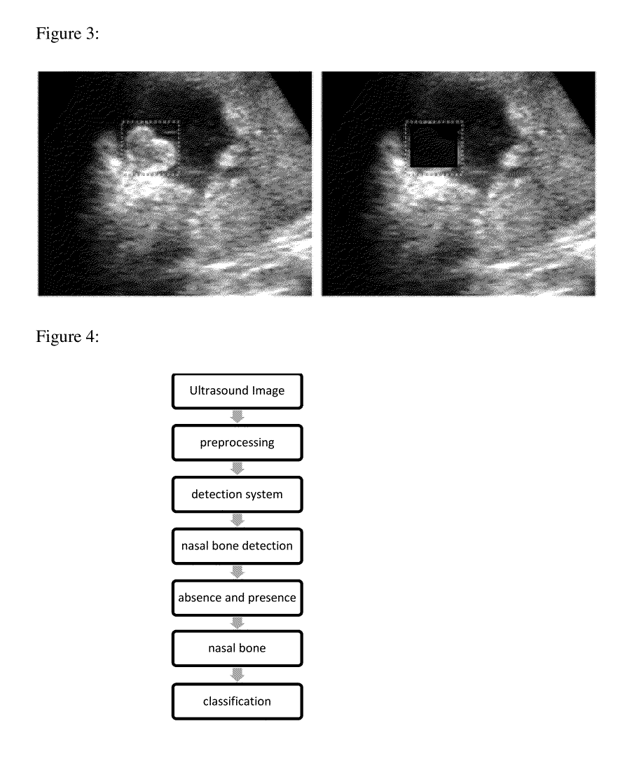

In the final step, the algorithm searches for genitalia-related features at each POI defined in step two. Genitalia will appear as either the “three-line sign” (females) or as a pair of elliptical or circular regions of similar size (males and sometimes females). The three-line sign can be detected by line tracking, and the circular regions can be detected using a method similar to the one for detecting the fetal skull described above. At each POI, the algorithm defines a region of a certain radius around the POI in which to search for genitalia-related features. The region with the most likely features is determined to be the location of the genitalia. This region can then be blacked out from the sonogram. The region determined to be the location of the genitalia will be automatically blacked out from the ultrasound image as shown in FIG. 3. The details of the three stages are described below.

### Stage 1:

The first stage involves identification of various anatomical features. This information is used for creating an anatomical map of the fetus, using which we can narrow down the area where the genitals may be situated. The anatomical features which we are trying to identify are skull, spine, femur and nasal bone. Different methods for identification of the various anatomical features have been described below.

### Spine Detection

Two independent techniques are employed to find an approximate estimate of the spine in the ultrasound image. The first technique makes use of the visual properties of fetal spine in US images to estimate the curve depicting the location of the spine. Iterative dilation is used which reduces the number of distinct regions, the segmented regions of the bone structures then join and form continuous chains. From these the longest chain will be the spine. This method works well for spinal bones because of the close proximity of the vertebrae with each other and the absence of other long anatomical features in an ultrasound image of a spine. The second technique is also applied on the segmented image. This step makes use of physical and physiological features of the spinal bones and thus acts to complement the curve estimated in the first technique. The features like size, shape, spatial proximity and orientation of the segmented regions are used to cluster them. Segmented regions are clustered into three classes based on the distance attributes using unsupervised K-means clustering algorithm. In the first step clustering of the segmented regions is done using the distances or proximity of each segmented region from its two nearest neighbours. Euclidean distance measure is used to calculate minimum distance between the boundaries of regions. In the second step we take the regions obtained from the previous step and compute their area. A bounding ellipse to each of the regions is found and their major and minor axes are obtained. The third attribute measures the orientation of the region with respect to its two nearest neighbors. The two features that we observe to detect the orientation of the spine are:

(1) There is a point of inflexion near the base of the spine. Thus, the spine tends to change from concave to convex or vice-versa. (2) The two rows of spinal bone tend to get close together near the base of the spine.

### Skull Detection

Different tissues in ultrasound images have various intensity ranges. In fetal head images, pixels corresponding to skull tissue have the highest intensity values. Thus, we can remove pixels that are not skull tissues by using an adaptive threshold. That is, pixels with intensity values less than the threshold are removed. The segmented skull structure produced by thresholding often appears like a white band with a width of several pixels. Before fitting the skull by a regular ellipse, thinning the band into one pixel width is preferred, since many redundant pixels can be eliminated. Using morphological operations in the distance field, edge thinning is achieved. The general elliptic equation is given by Equation. When A, B, C are not all zeros, these parameters can uniquely represent a conic curve. Ax²+Bxy+Cy²+Dx+Ey+F=0. This conic curve is ellipse, if and only if B²−4 AC<0. In practice, fetal skulls are often discontinuous and irregular in ultrasound images. We cannot obtain convincing results by only testing one pattern of five points, because there is no guarantee that the five points are all on the desired ellipse. The basic idea of Randomized Hough Transform (RHT) is to repeat the above procedure and accumulate results as a distribution. After a specified number of repetitions, we select the peak value with the largest distribution in the five dimensional parameter space as the final solution of A, B, C, D, E. Then we convert them into xc, yc, a, b, φ. The iterative randomized Hough transform (IRHT) improves the efficiency of RHT. Target region is a new technique introduced in IRHT. It is defined as the region where the ellipse is supposed to be found. In RHT, the target region is always the whole image and it does not change during the whole process. However, in IRHT, the target region shrinks gradually after every iteration. For example, the target region is initially the whole image, and in the first iteration, an ellipse is detected by RHT. The target region is shrunk from the whole image to a smaller rectangular region that encloses the detected ellipse. In the second iteration, RHT will be performed only in the adjusted target region. A better result is expected, because when the target region shrinks, more non-skull pixels are excluded from the target region. In other words, the interference from other types of tissues is decreased in the next iteration. The target region continues to shrink based on the result of its previous iteration until the size difference of the target regions between two iterations is less than 5%, and the detected ellipse in last iteration will be the final analysis result. In each repetition of RHT, three criteria are applied to test if the parameters A, B, C, D, E are valid or not. First, the conic curve must be an ellipse rather than a hyperbola or a parabola. In addition, the detected ellipse must be located within the target region. If a pattern satisfies all the criteria, it is accumulated in the parameter space; otherwise it is ignored. For example, RHT terminates when it exceeds the specified number of repetitions, which is set by user. But the accumulation often contains many invalid results. A large number of repetitions cannot guarantee the quality of the accumulation. IRHT, instead, terminates when the number of valid patterns exceeds a specified value. By doing so, it is guaranteed that a sufficient number of valid samples are used in the Hough Transform.

To further improve the efficiency of early elimination in the IRHT algorithm, the eccentricity of the detected ellipse, must be within a reasonable range. For example, if the eccentricity is close to 1, the ellipse will be shaped like a shuttle, which is obviously impossible for fetal skulls. Thus a user defined range of acceptable eccentricity is added to the IRHT algorithm to make it more efficient.

FIG. 4 shows a schematic diagram for nasal bone detection by a first embodiment. Prior to assess the absent of nasal bone, several image pre-processing techniques were implemented to trace the position of nasal bone due to random shape and position of embryo in ultrasound images. The characteristics that will be used to detect the nasal bone will be the 3 lines that show in the sonogram. The first 2 lines, which are proximal to the forehead, are horizontal and parallel to each other, resembling an equal sign. The top line represents the skin and the bottom line, which is thicker and more echogenic than the overlying skin, represents the nasal bone. A third line, which is almost in continuity with the skin but at a higher level, represents the tip of the nose.

Architecture of Map Matching with Normalized Grayscale Correlation Algorithm

Assume a given image S with matrix size P×Q and image T with matrix size M×N, where the dimensions of S are both larger than T. We proposed to call T as the Template Image Ti, j, and call the pattern in T as the Template Pattern, as well as calling S as the Search Image. Then, the output of S contains a subset image I where I and T are suitably similar in pattern and if such I exists; yield the location of I in S. The location of I in S will be referred to as the location of closest match, which will then been defined as the pixel index of the top-left corner of I in S. Let λ (i, j) be the correlation coefficient of T at location i, j of S, as defined in equation 3. The maximum value of k is set to value 1. Therefore, whenever the coordinate integers of (i, j) be such that λ (i, j) obtained the highest correlation coefficient. The algorithm will return i, j as the “closest match” in S.

### Image Correlation

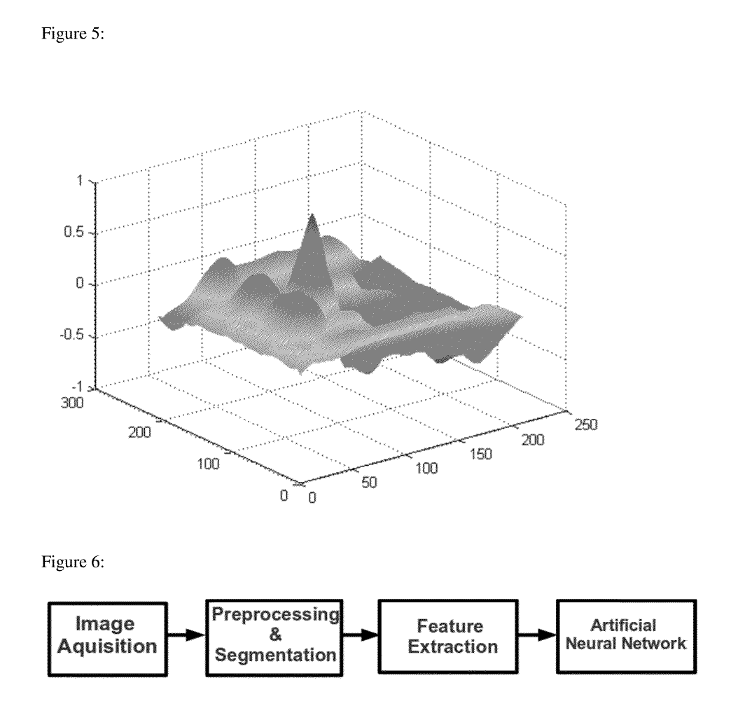

The normalized cross-correlation is calculated and will be displayed as a surface plot. The peak of the cross-correlation matrix occurs where the template image and target image are best correlated. However, algorithm must convert the image into grayscale before calculation of image correlation. Equation 4 computes the normalized cross-correlation of the matrices template and target. The target matrix must be larger than the template matrix in order to make the normalization meaningful. Nevertheless, the values of template cannot all be the same. The resulting matrix contains the correlation coefficients, which can range in value from −0 to 1.0. Where f=image, t′=mean of template and fu, v is the mean of f(x, y) in the region under the template. After calculated the image correlation, the next step of the developed algorithm is to convert the image correlation into surface plotting graph as shown in FIG. 5, which shows a surface topography map after convert the image correlation into surface plotting graph for the nasal detection according to the first embodiment. Based on the graph, we are able to obtain the maximum value of the image correlation which will be used for image classification of absence and presence nasal bone eventually. Based on the above method it has been found images having nasal bone present in them have maximum peak value above 0.35 as shown in FIG. 5.

FIG. 6 shows a schematic diagram for nasal bone detection by a second embodiment. The different steps of the nasal detection by the second embodiment are explained below.

### Preprocessing and Speckle Noise Reduction

Filtering is one of the common methods which is used to reduce the speckle noises. Speckle filtering consists of moving a kernel over each pixel in the image, doing a mathematical computation on the pixel values under the kernel and replacing the central pixel with the calculated value. The kernel is moved along the image one pixel at a time until the entire image has been covered. By applying the filter a smoothing effect is achieved and the speckle becomes less obtrusive. The speckle detection can be implemented with a median filter having a sliding window Sw×Sw. The Median filter is a simple one and removes pulse or spike noises. Pulse functions of less than one-half of the moving kernel width are suppressed

### Segmentation

There are many approaches available for ultrasound image segmentation including threshold methods, clustering learning, statistic model-based methods, and morphologic methods. Separating touching objects in an image is one of the more difficult image processing operations. Marker controlled watershed algorithm is applied to this problem. The watershed transform finds “catchment basins” and “watershed ridge lines” in an image by treating it as a surface where light pixels are high and dark pixels are low.

### Feature Extraction

In Transform domain Discrete Cosine transform technique and various multiresolution methods such as Daubechies 4 & Daubechies 6 were used for extracting parameter. The various parameters, which are used for the analysis, are mean, variance, skewness and kurtosis. Mean indicates the tendency to cluster around some particular value. The value, which characterizes its “width” or “variability” around the mean value, is the variance. Mean indicates the tendency to cluster around some particular value. The value, which characterizes its “width” or “variability” around the mean value, is the variance. The kurtosis is also a non-dimensional quantity. It measures the relative peakedness or flatness of a distribution to a normal distribution. (1) Discrete Cosine Transform: The discrete cosine transform finds use in many applications. The extracted DCT coefficients can also be used as a type of signature that is useful for recognition tasks each DCT coefficient can be viewed as representing a different feature dimension. The nasal bone detection process uses this approach. (2) Multilevel wavelet decomposition: A multiresolution representation provides a simple hierarchical framework for interpretating the image information. At different resolutions, the details of an image generally characterize different physical structures of the scene. At a coarse resolution, these details correspond to the larger structures which provide the image “context”. The discrete wavelets transform (DWT) decomposes an input signal into low and high frequency component using a Daubechies wavelet, which has important properties of orthogonality, linearity, and completeness. We can repeat the DWT multiple times to multiple-level resolution of different octaves. For each level, wavelets can be separated into different basis functions for image compression and recognition. Daubechies orthogonal wavelets D2-D20 are commonly used. The index number refers to the number N of coefficients. Wavelet functions are used here for the analysis are Daubechies 4 & Daubechies 6. The Daubechies wavelets are a family of orthogonal wavelets defining a discrete wavelet transform and characterized by a maximal number of vanishing moments. For each wavelet type, there is a scaling function (father wavelet) which generates an orthogonal multiresolution analysis. The Daubechies D4 transform has four wavelet and scaling function coefficients. The above mentioned features are used in training the neural network and later these are used by the trained neural network to find whether nasal bine is present or not.

### Neural Network Training

There are a number of efficient algorithms for the implementation of artificial neural network. Here Back Propagation (BP) algorithm is used for training. Training is carried out by iterative updation of weights based on the error signal. Here the negative gradient of a mean-squared error function is used. In the output layer, the error signal is the difference between the desired and actual output values, multiplied by the slope of a sigmoidal activation function. Then the error signal is back-propagated to the lower layers. BP is a descent algorithm which attempts to minimize the error at each iteration. The weights of the network are adjusted by the algorithm such that the error is decreased along a descent direction. Two parameters, learning rate (LR) and momentum factor (MF), are used for controlling the weight adjustment along the descent direction and for dampening oscillations.

To predict whether a new image displays Nasal bone or not, we need to have a training set from which the details of the new image can be predicted. To this effect we use a neural network, which will take in the inputs and train the network according to the image characteristics and creates two well defined boundaries for images with and without Down syndrome. Each image is first converted from RGB to gray-scale. The statistical parameters from large number of images are extracted and are then normalized. The normalized patterns are used in the training of Back Propagation Neural Network. Training set consists of (x1; t1); :::::,(xp; tp) p ordered pairs of n- and m-dimensional vectors, which are called the input and output patterns. When the input pattern xi from the training set is presented to this network, it produces an output Oi which is different from the target ti. What we want is to make Oi and ti identical for i=1; by using a learning algorithm. More precisely, we want to minimize the error function of the Network.

### Use of Trained Neural Network

After minimizing this function for the training set, new unknown input patterns are presented to the network and we expect it to interpolate. The network must recognize whether a new input vector is similar to learned patterns and produce a similar output. During the training process the numeral “0” is used to represent images without nasal bone and “1” is used to represent images having nasal bone. The training process create interconnection weights, these weight factors may be random and adjust them such that a sufficiently trained network can distinguish between normal and abnormal images. To create a network that can handle noisy inputs we have to train the network on both ideal and noisy vectors. For this purpose we alter the image, add noise and propagate the generated test vector file through the Back Propagation Neural Network. If the output of the neural network is between 0.5 and 1 distinguish the image as a normal image and give the test result as “Nasal Bone present”, and if the output of the neural network is between 0 and 0.5, distinguish the image as “Nasal Bone absent”.

### Stage 2:

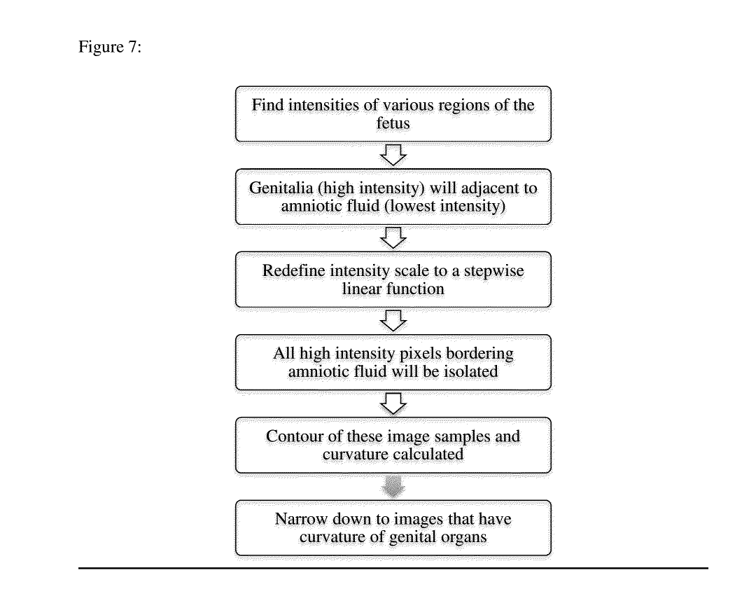

FIG. 7 shows a schematic diagram of stage 2 for the identification of the genitals. In the second step, the algorithm searches the region of interest and selects points of interest. It is assumed that the genitalia are visible in relief against the amniotic fluid, which is the lowest intensity uniform region in the ultrasound image. Thus, a distinguishing characteristic of pixels within the genitalia is their high intensity compared to pixels within the amniotic fluid. Because different ultrasound images will not have the same intensities, the histogram of the image is computed using a stepwise linear function with the lowest 10% intensity pixels receiving an intensity of 0 and the highest 10% receiving an intensity of 255. Each pixel in the image at a specified sampling space will be analyzed based on the pixels surrounding it. Pixels bordering the amniotic fluid (lowest intensity) will be selected as POIs. A second distinguishing characteristic of pixels within the genitalia is high curvature, due to the structure of the genitalia. The algorithm will sample the contours in the image and calculate the curvature of each POI on the contours. Those POIs whose curvature fails to exceed a certain threshold will be thrown out, leaving a new and reduced set of POIs.

### Stage 3:

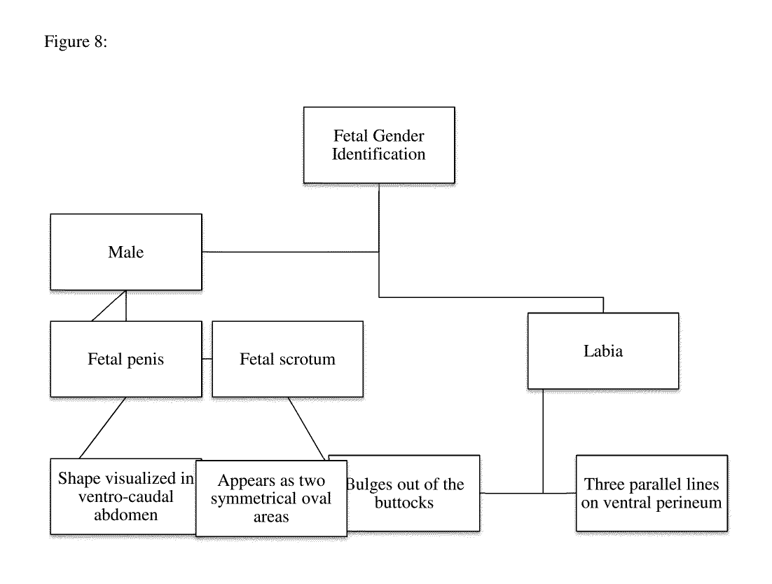

FIG. 8 shows a schematic diagram of stage 3 for the identification of the genitals. In the final step, the algorithm searches for genitalia-related features at each POI defined in step two. Genitalia will appear as either the “three-line sign” (females) or as a pair of elliptical or circular regions of similar size (males and sometimes females). The three-line sign can be detected by line tracking, and the circular regions can be detected using a method similar to the one for detecting the fetal skull described above. At each POI, the algorithm defines a region of a certain radius around the POI in which to search for genitalia-related features. The region with the most likely features is determined to be the location of the genitalia.

The algorithm employs a three-step process to locate the genitalia. In the first step, the algorithm searches for distinguishable characteristics of the fetus such as skull, femur bone, and nasal bone. Any detectable features are used to segment the fetus and narrow down the region of interest. In the second step, the algorithm performs a rough search over the region of interest and selects points of interest (POIs) based on the criteria described below. In the third step, the algorithm searches for the genitalia in the vicinity of each POI.

The skull and femur bones, if visible, are distinguishing characteristics of the fetus that can be used to reduce the region of interest. The skull appears as a relatively high-intensity ellipse or fragmented ellipse and can be detected using the iterative randomized Hough transform. The Hough transform is a feature extraction technique used in image analysis, computer vision, and digital image processing. The purpose of the technique is to find imperfect instances of objects within a certain class of shapes by a voting procedure. This voting procedure is carried out in a parameter space, from which object candidates are obtained as local maxima in a so-called accumulator space that is explicitly constructed by the algorithm for computing the Hough transform. The femur bone is also relatively high intensity and has a linear shape, making it detectable by line tracking. If the location and position of both the skull and one or both femur bones is known, the region of interest can be significantly reduced. Other features, such as the nasal bone, may also be detectable to further segment the fetus and narrow the region of interest.

In the second step, the algorithm searches the region of interest and selects points of interest. It is assumed that the genitalia are visible in relief against the amniotic fluid, which is the lowest intensity uniform region in the ultrasound image. Thus, a distinguishing characteristic of pixels within the genitalia is their high intensity compared to pixels within the amniotic fluid. Because different ultrasound images will not have the same intensities, the histogram of the image is computed using a stepwise linear function with the lowest 10% intensity pixels receiving an intensity of 0 and the highest 10% receiving an intensity of 255. Each pixel in the image at a specified sampling space will be analysed based on the pixels surrounding it. Pixels bordering the amniotic fluid (lowest intensity) will be selected as POIs. A second distinguishing characteristic of pixels within the genitalia is high curvature, due to the structure of the genitalia. The algorithm will sample the contours in the image and calculate the curvature of each POI on the contours. Those POIs whose curvature fails to exceed a certain threshold will be thrown out, leaving a new and reduced set of POIs.

In the final step, the algorithm searches for genitalia-related features at each POI defined in step two. Genitalia will appear as either the “three-line sign” (females) or as a pair of elliptical or circular regions of similar size (males and sometimes females). The three-line sign can be detected by line tracking, and the circular regions can be detected using a method similar to the one for detecting the fetal skull described above. At each POI, the algorithm defines a region of a certain radius around the POI in which to search for genitalia-related features. The region with the most likely features is determined to be the location of the genitalia. This region can then be blacked out from the sonogram image. The region determined to be the location of the genitalia will be automatically blacked out from the ultrasound image.

Another algorithm uses a sliding window based approach, in which it takes an ultrasound image, and detects and localizes the genitalia and outputs the image with the genitalia area blurred. The algorithm using the sliding window based approach, where the window size determined by the training data and is large enough to include the genitalia, to generate a number of features for each window. Then, a trained classifier recognizes whether the window contains genitalia or not, and provides a confidence level.

The algorithm for detection of genitalia is based on sliding a window across an ultrasound image at multi-scale and calculating features of different types and classifying each window as containing genitalia or non-genitalia. One other approach that has been utilized to increase the confidence in the results and reduce the number of false positives is to incorporate the context of the whole ultrasound image in the algorithm. This is achieved by using local and global features of the whole image to classify the ultrasound image as either containing the fetus or no-fetus (more details are below). The sliding window approach uses Support Vector Machine (SVMs) [Non Patent Citation 1] classifier that is trained to recognize the characteristics of genitalia, other possible classifiers than can be used for detection include neural networks [Non Patent Citation 2], naive Bayes classifiers [Non Patent Citation 3], boosted decision stumps [Non Patent Citation 4], etc. SVMs are supervised learning algorithms that recognize patterns in data and are used for classification and regression analysis. More formally, a support vector machine constructs a hyper-plane in a high- or infinite-dimensional space, which is then used for classification or other tasks. The classifier is trained by giving it different features of genitalia (positive training examples) and non-genitalia (negative training examples) images taken from different viewing angles and locations as well as of different gestation stages, genders, and patient shapes and sizes. The algorithm then tries to find a section of the live ultrasound image that contains genitalia by testing an overlapping window that moves over the image for features that match the training genitalia images.

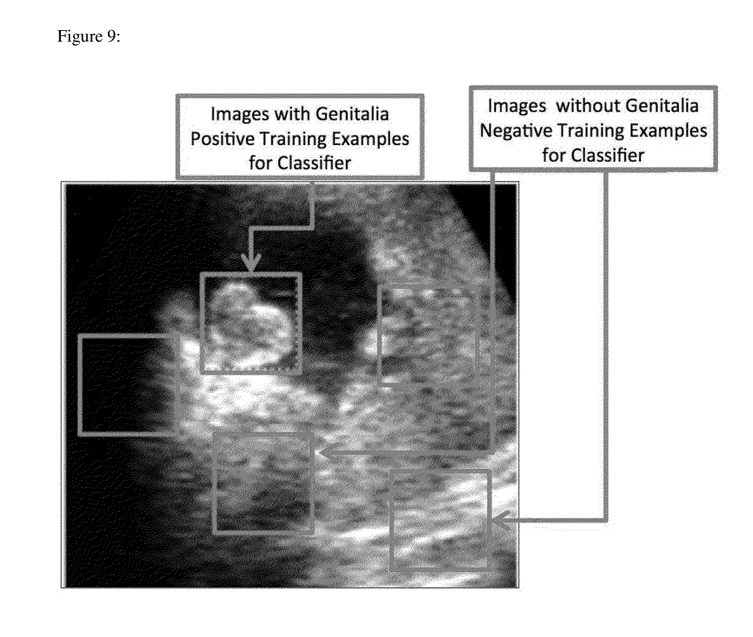

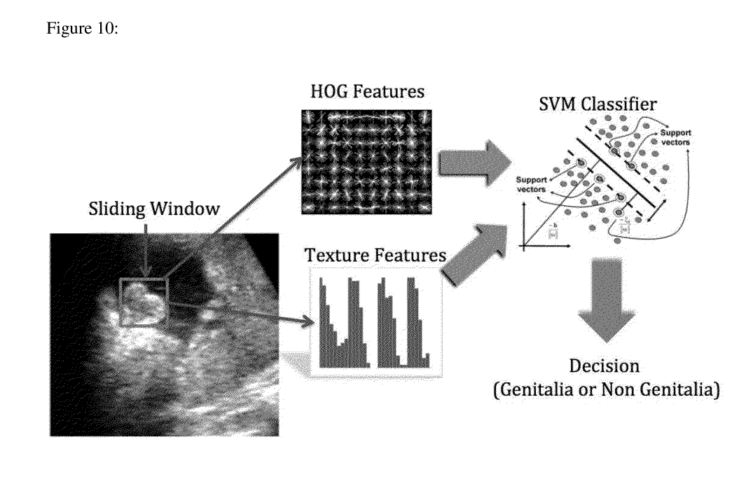

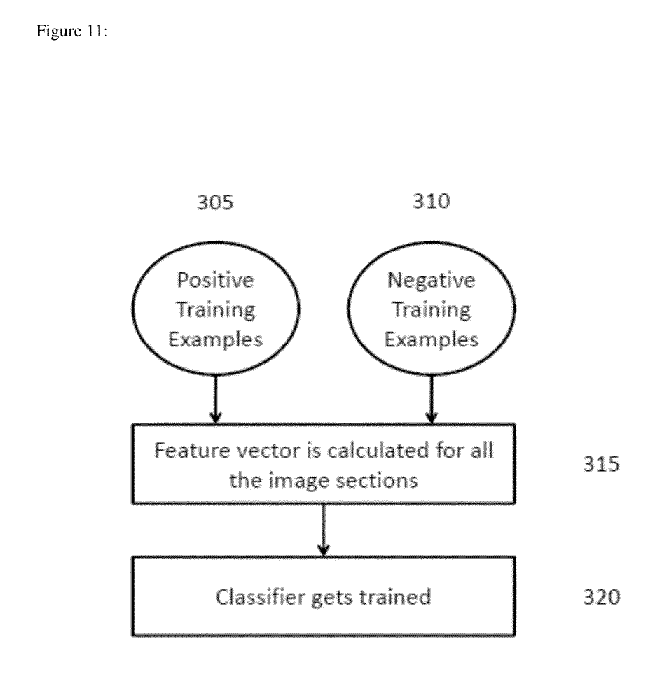

The technology could be in the form of software or hardware that can be embedded either directly into an ultrasound system or made available as an external hardware module. The image analysis for detection of genitalia is based on a sliding window approach and different features are calculated and concatenated. The features are Histogram of Oriented Gradients (HOG) [Non Patent Citation 5] and different texture features: co-occurrence matrix [Non Patent Citation 6], Gabor filter (Gabor filters are excellent band-pass filters for unidimensional signals in images and are popular texture features in image classification) [Non Patent Citation 7] and Local binary patterns (Local binary patterns are a type of texture features used for classification in computer vision) [Non Patent Citation 10]. HOG are feature descriptors used in computer vision for the purpose of object detection and recognition. The technique counts occurrences of gradient orientation in localized areas of an image. We are in the process of collecting a large dataset of ultrasound images and had a clinical expert mark a bounding box around where the genital area is located. Half of the images are used for training, and the rest for testing the algorithm. In the training images, the marked genitalia are split up into cells of 6×6 pixels. The algorithm then computes the gradient, a vector containing the x-derivative and y-derivative, at each point. A histogram is created for each cell, where the orientation of each pixel in that cell is plotted, weighted by the pixel's magnitude. Then a feature vector is created for that image, by concatenating/combining all of the histograms and all the texture features calculated. By random generation as shown in FIG. 9, images will be created of the same size from the ultrasound images, by using sections that don't overlap with the marked genitalia, and will undergo a similar process to create more feature vectors. The classifier is then given the feature vectors of the genitalia and non-genitalia, and plots them in N-dimensional space, where N is the size of the feature vector. The SVMs classifier then generates a hyper-plane that separates the images that contained genitals from the other images. FIG. 10 shows a schematic diagram of the ultrasound-imaging algorithm using the sliding window based approach.

When the code is running on a test image or an image obtained directly from an ultrasound system, it will try to look for genitalia of the same size range of the training genitalia. To search the entire image, the code will look through a sliding window, and use the above method for creating a feature vector for that window. It then tries to match that vector to the training set, gives a confidence level for that area, and then moves the window to the next section of the image, allowing for overlap. Then, the areas with a confidence level higher than our threshold are displayed. If there is more than one area marked, then the highest one is displayed, while the others are tried with a higher threshold value, since the chance of multiple genitalia (twins, triplets, etc.) is low, but still is a possibility. Besides blocking the windows with the genitalia with a fixed intensity image, other techniques tested are pixelization and blur.

One way to increase the confidence in the results and reduce the number of false positives is to incorporate the context of the whole ultrasound image in the algorithm. This is achieved by using local and global features of the whole image to classify the ultrasound image as either containing the fetus or no-fetus and in case of fetus images it gives the confidence of a possible area where the genitalia might be present. The local and global features used here are texture features (co-occurrence matrix, Gabor filter) and Zernike moments (Zernike moments are the mappings of an shape or image onto a set of complex Zernike polynomials and used as a feature) [Non Patent Citation 8] and SVMs classifier is trained with both positive and negative image examples. We have tested this framework on a bio-image benchmark and have achieved similar classification results as reported in the paper [Non Patent Citation 9]. The Non Patent Citation 9, describes a bioimage classification benchmark.

The performance evaluation of the algorithms for detection and classification is based on the following metrics: Detection rate vs. False positives/Image and Receiver Operator Characteristic (ROC) curve, which are calculated based on true positives, true negatives, false positives, and false negatives.

The existing ultrasound machines can be retrofitted with a tamper-resistant “Ultrasafe Ultrasound” box that contains capabilities for image processing and Internet connectivity. Our approach in this area is to develop a secure reversible data-hiding scheme for embedding the images with the genitalia blocked/blurred and only an authorized person with a key will be able to reverse the blocking and get the original images or video stream. This approach has been proposed for privacy protection in surveillance video [Non Patent Citation 11]. The Non Patent Citation 11, describes a framework of data hiding for privacy preservation in a video surveillance system. To protect the privacy of individuals in a surveillance video, the areas of selected individuals in videos are blurred, or re-rendered. The box would then run the code, and display the fetus with the genital regions uncovered. There is handshake style software that will constantly monitor the box and report tempering to ensure the box is being used appropriately.

The method 300 as described in FIG. 11 explains the training process. Positive and negative training examples are inputted. In the positive examples, the images only contain the genitalia. The negative examples are images of the same size as the positive examples, but do not contain any part of the genitalia, and are generated as described in FIG. 10. In 315, a feature vector is created for each image in both sets, using the HOG feature and texture features. In 320, the classifier gets the feature vectors, and plots them in N-dimensional space, where N is the length of the feature vector. The SVM classifier will then generate a hyper-plane that separates the feature vectors of genitalia from the feature vectors of other images.

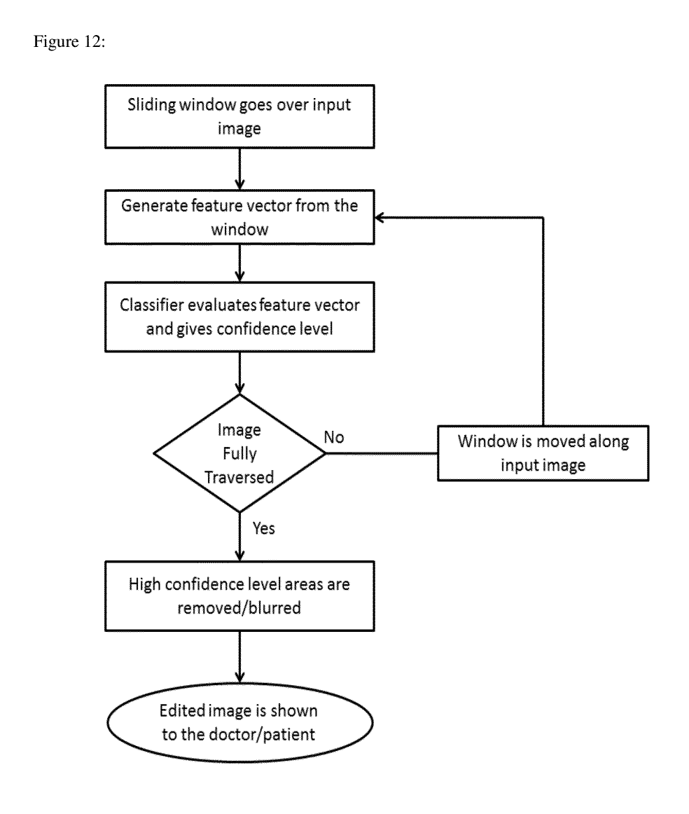

In FIG. 12, the method 400 describes the process by which an image from the ultrasound would be changed into an image that can be seen by the patient and doctor. The method uses a sliding window (block 405) that goes over the image. At each window, a feature vector is created using the HOG feature and texture features (block 410), as done with the training images. The feature vector is plotted by the SVM classifier, and the classifier returns whether the feature vector was on the same side as the feature vectors of genitalia, corresponding to the window containing genitalia, and a confidence level for that answer (block 415). Then, the algorithm checks to see if the image has been fully checked (block 420) and if not, moves the sliding window to check a new area, allowing for overlap (425). The method then goes back to block 410. After the image has been fully checked, the areas that had high confidence of containing genitalia will be removed or blurred (block 430). The images are then ready for the patient and doctor to view (block 435).

The structure of our algorithms lends itself well to efficient implementation on a compact and low-cost graphics processing unit (GPU) for real-time processing. While a GPU can be an inexpensive, low-power computing platform of high computational throughput, it is capable of these feats only when the algorithms map well to its overall architecture. Applications that map well are those that can be decomposed into independent blocks of computations that may be further decomposed into light-weighted threads. Such an application decomposition is well tailored to many cores of a GPU. In the described algorithm, the calculation of image features within a window is independent from the same calculation in another window in another part of the image. This means that the calculations needed for different windows can be performed all at once by assigning each window's calculations to different cores of the GPU. This matching of the application to the GPU architecture results in orders of magnitude acceleration of the application, with no changes to the algorithm itself.

The blurring/blocking of genitalia on live ultrasound images means processing successive frames of the ultrasound video. While the successive frames are expected to be slightly different because the operator may have moved the ultrasound probe or altered its orientation slightly, a great deal of similarity is still expected to be present between one frame to the next. Our algorithms also include methods to take advantage of this similarity between successive ultrasound frames (images). Using image registration techniques such as those based on optical flow and other image similarity measures, our algorithms determine the geometric change between two successive frames and use that information to transfer the findings of the ealier frame to next frame. This permits identifying points of interest, segmented features, and even information about genitalia much quicker with much less computation. When a substantial change occurs between successive frames, such as when the sonographer lifts the ultrasound probe off the skin and interrogates the fetus from a different viewing location and angle, the entire algorithm is run.

### CITATION LIST

### Non Patent Literature

[Non Patent Citation 1] C. Papageorgiou and T. Poggio. A trainable system for object detection. Intl. J. Computer Vision, 38(1):15-33, 2000.
[Non Patent Citation 2] Henry A. Rowley, Shumeet Baluja, and Takeo Kanade. Human face detection in visual scenes. In Advances in Neural Info. Proc. Systems, volume 8, 1995.
[Non Patent Citation 3] H. Schneiderman and T. Kanade. A statistical model for 3D object detection applied to faces and cars. In CVPR, 2000.
[Non Patent Citation 4] P. Viola and M. Jones. Robust real-time object detection. Intl. J. Computer Vision, 57(2):137-154, 2004.
[Non Patent Citation e 5] N. Dalal and B. Triggs. Histograms of Oriented Gradients for Human Detection. In CVPR, pages 886-893, 2005
[Non Patent Citation 6] R. M. Haralick, K. Shanmugam, and I. Dinstein, “Textural Features for Image Classification”, IEEE Transactions on Systems, Man, and Cybernetics, 1973, SMC-3 (6): 610-621
[Non Patent Citation 7] J. G. Daugman. Uncertainty relation for resolution in space, spatial frequency, and orientation optimized by two-dimensional visual cortical filters. Journal of the Optical Society of America A, 2(7):1160-1169, July 1985.
[Non Patent Citation 8] A. Khotanzad, and Y. H. Hong. “Invariant image recognition by Zernike moments.” Pattern Analysis and Machine Intelligence, IEEE Transactions on 12.5 (1990): 489-497.
[Non Patent Citation 9] L. Shamir, N. Orlov, D. M. Eckley, T. Macura, I. Goldberg; IICBU-2008—A proposed benchmark suite for biological image analysis, Medical & Biological Engineering & Computing, vol. 46, no. 9, pp. 943-947. Springer, 2008.
[Non Patent Citation 10] T. Ojala, M. Pietikainen, & T. Maenpaa. Multiresolution gray-scale and rotation invariant texture classification with local binary patterns. Pattern Analysis and Machine Intelligence, IEEE Transactions on, 24(7), 971-987. 20
[Non Patent Citation 11] J. K. Paruchuri, S. C. S. Cheung, & M. W. Hail (2009). Video data hiding for managing privacy information in surveillance systems. EURASIP Journal on Information Security, 2009, 7.

1. A method for detecting a fetus genitalia in an ultrasound image, comprising:
excluding regions from the ultrasound image, wherein the regions do not contain any feature of the fetus genitalia;
identifying points of interest (POIs) from remaining regions of the ultrasound image;
detecting the fetus genitalia from the POIs.

2. The method of claim 1, wherein the regions excluded contain features of the fetus selected from a group consisting of skull, femur bone and nasal bone.

3. The method of claim 1, wherein identifying POIs comprises selecting regions with a contrast above a threshold.

4. The method of claim 1, further comprising excluding POIs with curvatures below a threshold.

5. The method of claim 1, further comprising obscuring regions around the POIs from which the fetus genitalia is detected.

6. The method of claim 1, wherein detecting the fetus genitalia from the POIs comprises detecting features characteristic of labia, scrotum, penis, or a combination thereof.

7. The method of claim 1, wherein identifying POIs comprises selecting regions adjacent to pixels with the lowest 10% intensity among all pixels of the ultrasound image.

8. The method of claim 1, wherein detecting the fetus genitalia from the POIs comprises using a classification model.

9. The method of claim 1, further comprising training a classification model using ultrasound images that contain at least one feature of fetus genitalia.

10. The method of claim 9, further comprising training a classification model using ultrasound images that do not contain any feature of fetus genitalia.

11. The method of claim 1, wherein the classification model is a support vector machine.

12. The method of claim 1, wherein the classification model is a sliding window classifier.

13. A sonographic instrument comprising a physical processor and a physical memory having instructions recorded thereon, the instructions when executed by the physical processor implementing the method of any of claims 1-12.

14. The sonographic instrument of claim 13, further comprising a transducer configured to product an ultrasound wave, a sensor configured to receive echo of the ultrasound wave, and a display configured to display the ultrasound image.

15. A method for diagnosing anomaly in an ultrasound image, comprising:
obtaining the ultrasound image from a patient;
making a redacted image by obscuring at least one region of the ultrasound image;
making information of the redacted image available to the patient;
anonymizing the ultrasound image;
diagnosing the anomaly by analysing the anonymized ultrasound image;
making the diagnosis available to the patient;
wherein no information of the obscured region except the diagnosis is made available to the patient.

16. A method comprising detecting a feature in an ultrasound image prior to displaying the feature on a monitor in a vicinity of an ultrasound machine, and hiding the feature so as to prevent the feature from being displayed on the monitor, the method employing a sliding window algorithm.

17. The method of claim 16, further comprising retaining the feature that is hidden from viewing on the monitor for remote viewing.

18. The method of claim 16, further comprising exploiting similarity between frames of ultrasound images close in time to transfer findings from one frame to another frame.

19. The method of claim 16, further comprising a graphics processing unit acceleration for real-time implementation.
